(19) United States
(12) Patent Application Publication
(10) Pub. No.: US 2025/0197345 A1
(43) Pub. Date: Jun. 19, 2025

(54) SYNTHESIS OF DIGLYCOLAMIDES

(71) Applicant: Iowa State University Research Foundation, Inc., Ames, IA (US)

(72) Inventors: Long Qi, Ames, IA (US); Lun An, Ames, IA (US)

(21) Appl. No.: 18/978,966

(22) Filed: Dec. 12, 2024

Related U.S. Application Data
(60) Provisional application No. 63/610,840, filed on Dec. 15, 2023.

(51) Int. Cl. C07C 231/02; C22B 59/00

(52) U.S. Cl. CPC C07C 231/02; C22B 59/00

(57) ABSTRACT

A method of forming a diglycolamide includes heating a reaction solution including a diglycolic acid ester having the structure R⁵O—C(O)—CH(R³)—O—CH(R⁴)—C(O)—OR⁶, and an amine having the structure R¹—NH—R², to form the diglycolamide having the structure (R¹)(R²)N—C(O)—CH(R³)—O—CH(R⁴)—C(O)—N(R¹)(R²).

### STATEMENT OF GOVERNMENT SUPPORT

This invention was made with Government support under DE-AC02-07CH11358 awarded by the Department of Energy. The Government has certain rights in this invention.

### BACKGROUND

Diglycolamides (DGAs), a class of compounds of high interest for their ability to separate rare earth elements. DGAs are of interest due to their high degree of tunability allowing them to separate actinide and lanthanide series elements. DGAs are also of interest in fields of nuclear waste treatment and treatment of toxic or hazardous materials. DGAs can selectively separate elements such as rare earths that are difficult to separate with conventional methods due to their chemical similarity. DGAs have high affinity for trivalent metal ions and have structures that are easily modified. However, widespread use of DGAs is hindered by the cost associated with producing them. Further, DGA production typically requires the use of toxic reagents and produces toxic and/or hazardous byproducts.

### SUMMARY OF THE INVENTION

Various aspects of the present invention provide a method of forming a diglycolamide. The method includes heating a reaction solution including a diglycolic acid ester having the structure R⁵O—C(O)—CH(R³)—O—CH(R⁴)—C(O)—OR⁶. The reaction solution also includes an amine having the structure R¹—NH—R². The heating of the reaction solution forms the diglycolamide having the structure (R¹)(R²)N—C(O)—CH(R³)—O—CH(R⁴)—C(O)—N(R¹)(R²). The variables R⁵ and R⁶ are independently chosen from a substituted or unsubstituted (C₁-C₃₀) hydrocarbyl optionally interrupted by 0, 1, 2, or 3 groups independently chosen from —O— and —S—. The variables R¹ and R² are independently chosen from a substituted or unsubstituted (C₁-C₃₀) hydrocarbyl optionally interrupted by 0, 1, 2, or 3 groups independently chosen from —O— and —S—, or wherein R¹ and R² together form a ring containing the nitrogen atom to which they are attached and R¹ and R² are together a (C₄-C₁₂) hydrocarbylene optionally interrupted by 0, 1, 2, or 3 groups independently chosen from —O— and —S—. The variables R³ and R⁴ are independently chosen from —H and a substituted or unsubstituted (C₁-C₃₀) hydrocarbyl optionally interrupted by 0, 1, 2, or 3 groups independently chosen from —O— and —S—.

Various aspects of the present invention provide a method of forming a diglycolamide. The method includes heating a reaction solution in a continuous flow reactor at a temperature of 180° C. to 280° C. The reaction solution includes a diglycolic acid ester having the structure R⁵O—C(O)—CH₂—O—CH₂—C(O)—OR⁶. The reaction solution also includes an amine having the structure R¹—NH—R². The heating of the reaction solution forms the diglycolamide having the structure (R¹)(R²)N—C(O)—CH₂—O—CH₂—C(O)—N(R¹)(R²). The variables R¹, R², R⁵, and R⁶ are independently chosen from (C₁-C₁₀)alkyl. The variables R¹ and R² are independently chosen from (C₁-C₁₀)alkyl, or wherein R¹ and R² together form a ring containing the nitrogen atom to which they are attached and R¹ and R² are together a (C₄-C₁₂)hydrocarbylene

Various aspects of the present invention provide a system for performing the method of forming a diglycolamide. The system includes one or more pumps. The system includes a mixer to combine materials flowing from the one or more pumps and to form the reaction solution. The system includes a continuous flow reactor that receives the reaction solution from the mixer. The system also includes an analyzer that receives the reaction solution from the continuous flow reactor.

Various aspects of the present invention provide a system for performing the method of forming a diglycolamide. The system includes a batch reactor that contains the reaction solution while the reaction solution is heated to form the diglycolamide.

Various aspects of the present method of forming a diglycolamide and system for performing the same have various advantages over other methods of forming diglycolamides. For example, in various aspects, the present method of forming a diglycolamide can be performed without catalysts. In various aspects, the present method of forming a diglycolamide can be performed without additives, such that diglycolic acid ester, amine, and any solvent are the only components of the reaction solution. In various aspects, the present method of forming a diglycolamide can use and/or produce less toxic or hazardous materials than other methods of forming diglycolamides.

In various aspects, the method is a continuous flow process for forming the diglycolamide. As compared to a batch process, the continuous flow process can be more easily scaled up, can require less manual labor, can produce less waste, can cost less per amount of diglycolamide produced, can be safer, can be more easily regulated (e.g., temperature, concentration, and ratios of reactants can be more easily controlled and adjusted), can be more quickly and easily optimized (e.g., for high conversion and yield), can be more easily automated, or a combination thereof.

The wide application of DGAs has been limited by the current lab-scale batch synthesis, which is performed in round-bottomed flasks, test tubes, or closed vessels. Batch reactions usually require significant manual labor, as the reaction components from each step must be processed separately. On the other hand, in continuous flow synthesis, the process is controlled by a pre-set reaction sequence and allows it to run continuously under well-regulated conditions. This can save time and reduce waste and cost. Compared to batch processes, reaction parameters such as reactant equivalents, mixing, temperature, time, and concentration can be regulated in flow reactors. Furthermore, automated reaction optimization and discovery are feasible when coupling flow reaction systems with artificial intelligence/machine learning (AI/ML) algorithms, enabling simultaneous cooptimization of multiple reaction parameters for intelligent and autonomous manufacturing of chemicals and materials. Flow chemistry also provides solutions to batch reactions that are not feasible due to poor heat and mass transfer. Regarding DGA synthesis, all reported methods are based on batch reaction and require pre-activating diglycolic acid to diglycolic chloride to promote the amide bond formation. The pre-activation step requires additional synthetic work and handling of toxic reagents, such as thionyl chloride. In this context, developing a more environmentally-friendly and atom-economical method for DGA ligand synthesis is highly desirable, which should also hold the advantages of autonomous operation and facile scale-up. Various aspects of the present method provide an innovative method towards sustainable DGA synthesis under continuous flow setup using low-cost alkyl diglycolate diester and dialkylamine, wherein the method is readily scalable for mass production. In various aspects, the continuous flow process is compatible with real-time monitoring using online instruments (e.g., IR analysis) for continuous observation.

### BRIEF DESCRIPTION OF THE FIGURES

The drawings illustrate generally, by way of example, but not by way of limitation, various aspects of the present invention.

FIG. 1 illustrates structures and respective conversions of various diglycolamides formed via continuous synthesis, in accordance with various aspects.

FIG. 2 illustrates a diagram of a system for continuous synthesis, in accordance with various aspects.

FIG. 3A illustrates conversion versus temperature for continuous synthesis of N,N,N′,N′-tetrabutyl diglycolamide (TBDGA), in accordance with various aspects.

FIG. 3B illustrates conversion versus temperature for continuous synthesis of tetra-n-hexyl diglycolamide (THDGA), in accordance with various aspects.

FIG. 3C illustrates conversion versus temperature for continuous synthesis of tetra-n-octyl diglycolamide (TODGA), in accordance with various aspects.

FIG. 3D illustrates conversion versus temperature for continuous synthesis of N,N′-dimethyl-N,N′-dioctyl diglycolamide (DMDODGA), in accordance with various aspects.

FIG. 3E illustrates conversion versus temperature for continuous synthesis of tetra(2-ethylhexyl) diglycolamide (TEHDGA), in accordance with various aspects.

FIG. 3F illustrates conversion versus temperature for continuous synthesis of di(piperidin-1-yl) diglycolamide (DPipDGA), in accordance with various aspects.

FIG. 3G illustrates conversion versus temperature for continuous synthesis of di(pyrrolidin-1-yl) diglycolamide (DPyrDGA), in accordance with various aspects.

FIG. 3H illustrates conversion versus temperature for continuous synthesis of tetra(iso-butyl) diglycolamide (T′BDGA), in accordance with various aspects.

FIG. 4 illustrates structures of various diglycolamides formed via continuous synthesis, in accordance with various aspects.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo (carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” can refer to a functional group derived from a straight chain, branched, or cyclic hydrocarbon, and can be alkyl, alkenyl, alkynyl, aryl, cycloalkyl, acyl, or any combination thereof.

### Method of Forming a Diglycolamide.

Various aspects of the present invention provide a method of forming a diglycolamide. The method includes heating a reaction solution, such as in a batch or continuous reactor. The reaction solution includes a diglycolic acid ester having the structure R⁵O—C(O)—CH(R³)—O—CH(R⁴)—C(O)—OR⁶. The reaction solution also includes an amine having the structure R¹—NH—R². The heating of the reaction solution forms the diglycolamide having the structure (R¹)(R²)N—C(O)—CH(R³)—O—CH(R⁴)—C(O)—N(R¹)(R²). The variables R⁵ and R⁶ can be independently chosen from a substituted or unsubstituted (C₁-C₃₀)hydrocarbyl optionally interrupted by 0, 1, 2, or 3 groups independently chosen from —O— and —S—. The variables R¹ and R² can be independently chosen from a substituted or unsubstituted (C₁-C₃₀)hydrocarbyl optionally interrupted by 0, 1, 2, or 3 groups independently chosen from —O— and —S—, or R¹ and R² can together form a ring containing the nitrogen atom to which they are attached and R¹ and R² are together a (C₄-C₁₂)hydrocarbylene optionally interrupted by 0, 1, 2, or 3 groups independently chosen from —O— and —S—. The variables R³ and R⁴ can be independently chosen from —H and a substituted or unsubstituted (C₁-C₃₀)hydrocarbyl optionally interrupted by 0, 1, 2, or 3 groups independently chosen from —O— and —S—.

The variables R⁵ and R⁶ can be independently chosen from a substituted or unsubstituted (C₁-C₃₀)hydrocarbyl optionally interrupted by 0, 1, 2, or 3 groups independently chosen from —O— and —S—. The variables R⁵ and R⁶ can be independently chosen from (C₁-C₃₀)hydrocarbyl. The variables R⁵ and R⁶ can be independently chosen from (C₁-C₁₀)alkyl (e.g., straight chain or branched (C₁-C₁₀)alkyl). The variables R⁵ and R⁶ can be independently chosen from methyl, ethyl, propyl, (C₄)alkyl, (C₅)alkyl, (C₆)alkyl, (C₇)alkyl, (C₈)alkyl, (C₉)alkyl, and (C₁₀)alkyl. The variables R⁵ and R⁶ can be —CH₃.

The variables R³ and R⁴ can be independently chosen from —H and a substituted or unsubstituted (C₁-C₃₀)hydrocarbyl optionally interrupted by 0, 1, 2, or 3 groups independently chosen from —O— and —S—. The variables R³ and R⁴ can be independently chosen from —H and (C₁-C₃₀)hydrocarbyl. The variables R³ and R⁴ can be independently chosen from —H and (C₁-C₁₀)alkyl (e.g., straight chain or branched (C₁-C₁₀)alkyl). The variables R³ and R⁴ can be —H. In various aspects, the diglycolic acid ester is dimethyl 2,2′-oxydiacetate.

The variables R¹ and R² can be independently chosen from a substituted or unsubstituted (C₁-C₃₀)hydrocarbyl optionally interrupted by 0, 1, 2, or 3 groups independently chosen from —O— and —S—, or R¹ and R² together can form a ring containing the nitrogen atom to which they are attached and R¹ and R² are together a (C₄-C₁₂)hydrocarbylene optionally interrupted by 0, 1, 2, or 3 groups independently chosen from —O— and —S—. The variables R¹ and R² can be independently chosen from (C₁-C₃₀)hydrocarbyl. The variables R¹ and R² can be independently chosen from (C₁-C₁₀)alkyl. The (C₁-C₁₀)alkyl can be a straight chain or branched (C₁-C₁₀)alkyl groups. The variables R¹ and R² can be independently chosen from methyl, ethyl, propyl, (C₄)alkyl (e.g., n-butyl or iso-butyl), (C₅)alkyl, (C₆)alkyl, (C₇)alkyl, (C₈)alkyl (e.g., n-octyl or 2-ethylhexyl), (C₉)alkyl, and (C₁₀)alkyl. The variables R¹ and R² can together be chosen from (C₄)hydrocarbylene (i.e., —CH₂—CH₂—CH₂—CH₂—), (C₅)hydrocarbylene, (C₆)hydrocarbylene, (C₇)hydrocarbylene, (C₈)hydrocarbylene, (C₉)hydrocarbylene, (C₁₀)hydrocarbylene, (C₁₁)hydrocarbylene, and (C₁₂)hydrocarbylene. In various aspects, R¹ and R² together with the nitrogen atom to which they are attached can form a pyrrolidine ring (e.g., R¹ and R² together are (C₄)hydrocarbylene) or piperidine ring (e.g., R¹ and R² together are (C₅)hydrocarbylene).

In various aspects, the amine can be chosen from dibutylamine, dihexylamine, dioctylamine, methyloctylamine, di(2-ethylhexyl)amine, pyrrolidine, and piperidine. In various aspects, the diglycolamide can be chosen from N,N,N′,N′-tetrabutyl diglycolamide (TBDGA), tetra-n-hexyl diglycolamide (THDGA), tetra-n-octyl diglycolamide (TODGA), N,N′-dimethyl-N,N′-dioctyl diglycolamide (DMDODGA), tetra(2-ethylhexyl) diglycolamide (TEHDGA), di(piperidin-1-yl) diglycolamide (DPipDGA), di(pyrrolidin-1-yl) diglycolamide (DPyrDGA), and tetra(iso-butyl) diglycolamide (TⁱBDGA).

The reaction solution can have any suitable molar ratio of the amine to the glycolic acid, such as at least 2:1, or 2:1 to 10:1, 3:1 to 5:1, or less than or equal to 10:1 and greater than or equal to 2:1 and less than, equal to, or greater than 2.2:1, 2.4:1, 2.6:1, 2.8:1, 3:1, 3.2:1, 3.4:1, 3.6:1, 3.8:1, 4:1, 4.2:1, 4.4:1, 4.6:1, 4.8:1, 5:1, 5.2:1, 5.4:1, 5.6:1, 5.8:1, 6:1, 6.2:1, 6.4:1, 6.6:1, 6.8:1, 7:1, 7.2:1, 7.4:1, 7.6:1, 7.8:1, 8:1, 8.5:1, 9:1, or 9.5:1.

The reaction solution can further include an organic solvent. The organic solvent can be any one or more suitable organic solvents. The organic solvent can have a boiling point in the range of 100° C. to 300° C., or 130° C. to 240° C., or less than or equal to 300° C. and greater than or equal to 100° C. and less than, equal to, or greater than 120° C., 125, 130, 135, 140, 145, 150, 155, 160, 165, 170, 175, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 250, 260, 270, 280, or 290° C. The organic solvent can include toluene, 1,4-dioxane, ethylene glycol, DMF (dimethylformamide), ACN (acetonitrile), xylene, anisole, trifluoromethyl anisole, kerosene, or a combination thereof. The organic solvent can include an aprotic organic solvent. The diglycolic acid ester can have any suitable concentration in the reaction solution, such as a concentration of 0.01 M to 10 M, or 0.5 M to 1.5 M, or less than or equal to 10 M and greater than or equal to 0.01 M and less than, equal to, or greater than 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, or 9 M.

The reaction solution can further include one or more additives. The additive can be insoluble in the reaction during the heating of the reaction solution and during the formation of the diglycolamide. The additive can include SiO₂, mesoporous silica, Al₂O₃, a material including a Lewis acid site (e.g., zeolites such as zeolites having an aluminum framework; metal oxides such as alumina (Al₂O₃); a clay; or a compound containing boron, titanium, or zirconium), a material including a Bronsted acid site (e.g., zeolites such as zeolites having aluminum incorporates into their structure, silica-alumina, a metal-organic framework (MOF), a solid acid wherein a bridging hydroxyl group (—OH) can act as a proton donor), a homogenous acid material, a heterogeneous acid material, or a combination thereof. The one or more additives can form any suitable proportion of the combination of the diglycolic acid ester and the one or more additives in the reaction solution, such as 0 wt % to 95 wt %, or 5 wt % to 90 wt %, or 10 wt % to 80 wt %, or less than or equal to 95 wt % and greater than or equal to 0 wt % and less than, equal to, or greater than 1 wt %, 2, 3, 4, 5, 6, 8, 10, 12, 14, 16, 18, 20, 25, 30, 35, 40, 45, 50, 55, 60, 65, 70, 75, 80, 82, 84, 86, 88, 90, 91, 92, 93, or 94 wt % of the combination of the diglycolic acid ester and the one or more additives in the reaction solution. The one or more additives can have any suitable weight ratio to the diglycolic acid ester, such as a ratio of 0.01:1 to 2:1, or less than or equal to 2:1 and greater than or equal to 0.01:1 and less than, equal to, or greater than 0.05:1, 0.1:1, 0.15:1, 0.2:1, 0.25:1, 0.3:1, 0.4:1, 0.5:1, 0.6:1, 0.7:1, 0.8:1, 0.85:1, 0.9:1, 0.95:1, 1:1, 1.05:1, 1.1:1, 1.15:1, 1.2:1, 1.3:1, 1.4:1, 1.5:1, 1.6:1, 1.7:1, 1.8:1, or 1.9:1. In various aspects, the reaction solution can be substantially free of the one or more additives. In various aspects, the reaction solution can be substantially free of materials other than the diglycolic acid ester, the amine, any of the diglycolamide formed from the diglycolic acid ester and the amine, and one or more optional solvents. In various aspects, the reaction solution can be substantially free of materials other than the diglycolic acid ester, the amine, any of the diglycolamide formed from the diglycolic acid ester and the amine, one or more optional solvents, and the one or more additives.

The method can have any suitable yield of the diglycolamide from the diglycolic acid ester, such as a yield of 50% to 100%, 90% to 100%, or less than or equal to 100% and greater than or equal to 50% and less than, equal to, or greater than 55%, 60, 65, 70, 75, 80, 82, 84, 86, 88, 90, 91, 92, 93, 94, 95, 96, 97, 98, 99, 99.9, or 99.99%. The method can have any suitable conversion of the diglycolic acid ester, such as a conversion of 50% to 100%, 90% to 100%, or less than or equal to 100% and greater than or equal to 50% and less than, equal to, or greater than 55%, 60, 65, 70, 75, 80, 82, 84, 86, 88, 90, 91, 92, 93, 94, 95, 96, 97, 98, 99, 99.9, or 99.99%.

In various aspects, the method can be a batch method. For example, the reaction solution can be heated in a batch reactor during the heating such that substantially no reaction fluid flows in or out of the batch reactor during the heating.

In other aspects, the method can be a continuous method. The reaction solution can be heated in a continuous flow reactor during the heating, wherein the reaction solution continuously flows into and out of the continuous flow reactor during the heating. The continuous flow reactor can be any suitable continuous flow reactor, such as a tubular reactor or a coil reactor.

The reactor can include a heater to generate heat for the heating of the reaction solution. The heating can include heating the reaction solution to a treatment temperature for a treatment duration. The treatment temperature can be any suitable treatment temperature that accomplishes formation of the diglycolamide, and can include a temperature of 100° C. to 300° C., or 180° C. to 280° C., or 180° C. to 240° C., or less than or equal to 300° C. and greater than or equal to 100° C. and less than, equal to, or greater than 120° C., 140, 150, 160, 170, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 250, 260, 270, 280, or 290° C. The treatment duration can be any suitable duration which, at the treatment temperature used, accomplishes formation of the diglycolamide, such as a duration of 1 min to 24 h, or 1 h to 12 h, 1 h to 3 h, or less than or equal to 24 h and greater than or equal to 1 min and less than, equal to, or greater than 2 min, 4, 6, 8, 10, 15, 20, 25, 30, 35, 40, 45, 50, 55, 65, 70, 75, 80, 85, 90, 95, 100, 105, 110, 115 min, 2 h, 2.5, 3, 3.5, 4, 4.5, 5, 6, 7, 8, 9, 10, 11, 12, 14, 16, 18, 20, or 22 h. In a continuous flow reactor, the treatment duration can be the residence time of the reaction solution in the continuous flow reactor. The pressure can be maintained at any suitable pressure during the treatment duration. The pressure can be ambient pressure. The pressure can be elevated pressure. The pressure can be 10 psi to 10,000 psi, or less than or equal to 10,000 psi and greater than or equal to 10 psi and less than, equal to, or greater than 11 psi, 12, 13, 14, 15, 16, 17, 18, 19, 20, 22, 24, 26, 28, 30, 35, 40, 45, 50, 60, 70, 80, 90, 100, 150, 200, 250, 300, 400, 500, 750, 1,000, 1,500, 2,000, 2,500, 5,000, or 7,500 psi.

The reaction solution can be substantially free of halides, such as chlorides and bromides. The reaction solution can be substantially free of thionyl halides, such as thionyl chloride. The reaction solution can be substantially free of an acid halide of the diglycolic acid ester (i.e., having the structure X—C(O)—CH(R³)—O—CH(R⁴)—C(O)—X wherein X is halide). The reaction solution can be substantially free of the acid form of the diglycolic acid ester (i.e., having the structure HO—C(O)—CH(R³)—O—CH(R⁴)—C(O)—OH).

The method can further include using the diglycolamide as a ligand to perform an extraction and/or separation of rare earth metals, nuclear waste, toxic or hazardous materials, or a combination thereof. For example, the diglycolamide can be added to an ore or mixture containing rare earth metals, nuclear waste, toxic or hazardous materials, or a combination thereof, and the ligand can selectively bind to one or more materials of interest in the ore or mixture (e.g., the diglycolamide can selectively or preferentially bind to one or more rare earth metals, a material emitting nuclear radiation, or to one or more toxic or hazardous materials), allowing the ligand-bound materials to be separated from the remainder of the ore or mixture.

### System for Forming a Diglycolamide.

In various aspects the present invention provide a system for performing the method of forming a diglycolamide described herein. The system can include one or more pumps. The system can include a mixer to combine materials flowed from the one or more pumps (e.g., the diglycolic acid ester, the amine, any solvent, and any additive) and to form the reaction solution. The system can include a continuous flow reactor that receives the reaction solution from the mixer. The continuous flow reactor includes a heater that heats the reaction solution to form the diglycolamide therefrom. The system can include an analyzer that receives the reaction solution from the continuous flow reactor. The analyzer can be an infrared spectrometer.

The system can include a processor that receives data from the analyzer and that controls the pumps and the continuous flow reactor (e.g., controls flow rate through the reactor and/or temperature of the reactor). The processor can improve or optimize flow rate and reaction. For example, the processor can determine the yield and percent conversion for a given temperature and given flow rates of the pumps by using data received from the analyzer, and the processor can vary the temperature and flow rates. By analyzing a variety of flow rates and temperatures, over time the processor can determine the conditions needed to increase both the conversion and the yield. In various aspects, the processor can maximize both the conversion of the diglycolic acid ester and the yield of the diglycolamide.

Various aspects of the present invention provide a system for performing the method of forming a diglycolamide. The system can include a batch reactor that contains the reaction solution while the reaction solution is heated to form the diglycolamide. The system can include one or more pumps. The system can include a mixer to combine materials flowed from the one or more pumps (e.g., the diglycolic acid ester, the amine, any solvent, and any additive) and to form the reaction solution. The batch reactor can receive the reaction solution from the mixer. The batch reactor can include a heater that heats the reaction solution to form the diglycolamide therefrom. The system can include an analyzer that receives the reaction solution from the batch reactor. The analyzer can be an infrared spectrometer.

### EXAMPLES

Various aspects of the present invention can be better understood by reference to the following Examples, which are offered by way of illustration. The present invention is not limited to the Examples given herein.

### Example 1. Synthesis of DGA Under Batch Conditions

The synthesis of DGA from dimethyl 2,2′-oxydiacetate (acid-catalyzed esterification from diglycolic acid and methanol) with dibutyl amine as the representative dialkylamine was optimized under batch conditions. To a 25 mL pressure tube equipped with a magnetic stir bar was added dimethyl 2,2′-oxydiacetate (162.1 mg, 1.0 mmol, 1.0 eq.), dibutyl amine (517.0 mg, 4.0 mmol, 4.0 eq.) and xylene (isomer mixtures, 1 mL). The pressure tube was sealed with a Teflon cap, and the reaction mixture was heated at the indicated temperature for 8 h before cooling to room temperature. Xylene and excess dibutyl amine were removed under reduced pressure. The conversion of dimethyl 2,2′-oxydiacetate 1 was determined by ¹H NMR in CDCl₃. Screening different organic solvents demonstrated that xylene was a superior solvent for this transformation (Table 1, entries 1-5), resulting in 82% conversion of 1 at 180° C. (Table 1, entry 5). Attempts to have better conversion and yields were carried out by adding additives in wt. % relative to the weight of the dimethyl 2,2′-oxydiacetate 1. The highest conversion was obtained with SiO₂ as an additive at 200° C. (Table 1, entry 8). Besides xylene, anisole can also be used as a solvent in our system, resulting in comparable conversion of 1 without SiO₂ additive (Table 1, entry 11). The reaction conditions used were 1 (1 mmol, 1.0 equiv.), 2 (2-4 mmol, 2-4 equiv.), solvent (1 mL, 1.0 M) stirred in a sealed pressure tube for 8 h. In Entry 1, diethyl diglycolate was detected in quantitative yield. Yields were determined by ¹H-NMR using mesitylene as an internal standard.

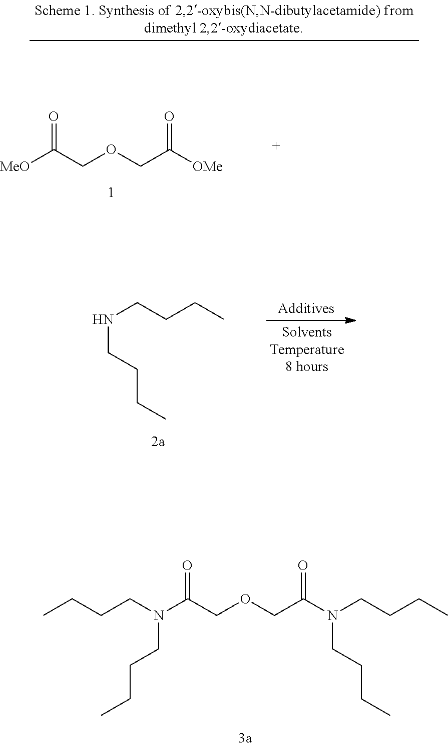

TABLE 1
Reaction parameters for the batch synthesis
of 2,2′-oxybis(N,N-dibutylacetamide).

| | | | Temper- | | 2a, Con- | 3a, |
|---|---|---|---|---|---|---|
| | | | ature | 2a | version | Yield |
| Entry | Additive | Solvent | (° C.) | (equiv.) | (%) | (%) |
| 1 | None | EtOH | 120 | 4.0 | 100 | 0 |
| 2 | None | DMF | 120 | 4.0 | 50 | 0 |
| 3 | None | ACN | 120 | 4.0 | 0 | 0 |
| 4 | None | Xylene | 120 | 4.0 | 0 | 0 |
| 5 | None | Xylene | 180 | 4.0 | 82 | 38 |
| 6 | None | Xylene | 200 | 4.0 | 100 | 55 |
| 7 | SBA-15 (50 | Xylene | 200 | 4.0 | 100 | 75 |
| | wt. %) | | | | | |
| 8 | SiO₂ (50 wt. | Xylene | 200 | 4.0 | 100 | 90 |
| | %) | | | | | |
| 9 | Al₂O₃ (30 wt. | Xylene | 200 | 4.0 | 100 | 55 |
| | %) | | | | | |
| 10 | Al₂O₃ (50 | Xylene | 200 | 4.0 | 100 | 36 |
| | wt. %) | | | | | |
| 11 | None | Anisole | 200 | 4.0 | 100 | 80 |

### Example 2. Batch Synthesis of DGAs with Variation of Starting Materials

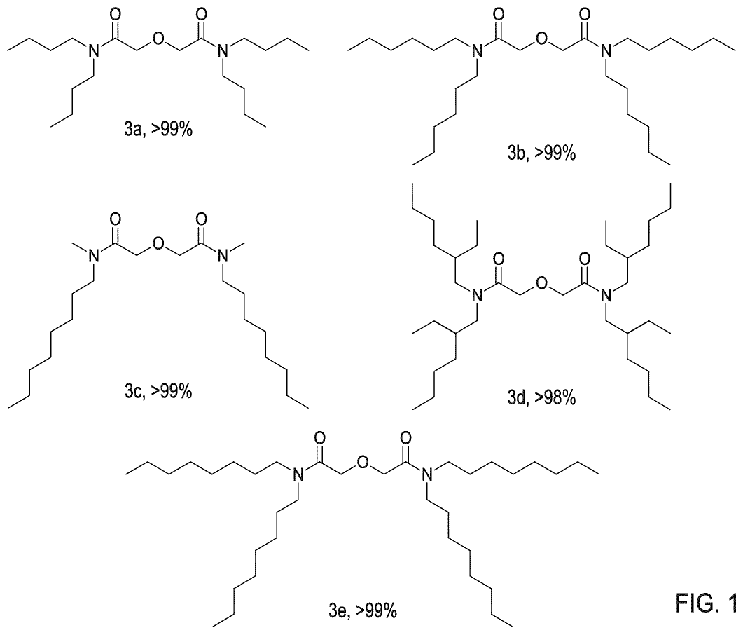

Scheme 2 illustrates the formation of diglycolamide 3 from diglycolic acid ester 1 and amine 2. The same reactor setup was used as described in Example 1. The same starting conditions were used as determined as optimal in Example 1. The reaction proceeded smoothly with various dialkyl amines. FIG. 1 illustrates structures and respective conversions of the diglycolamides formed via the continuous synthesis, which included TBDGA (3a), THDGA (3b), DMDODGA (3c), TEHDGA (3d), and TODGA (3e). The reaction was conducted at 200° C. for 8 hours with 50 wt % of SiO₂ added.

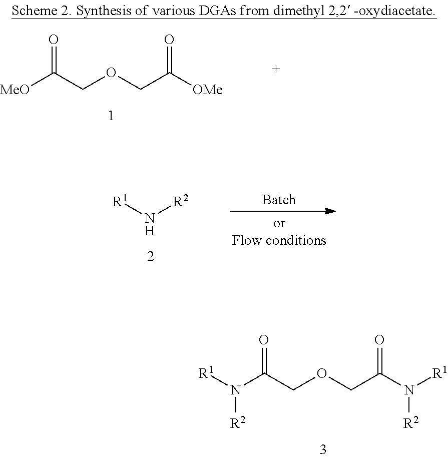

### Example 3. Reaction Optimization of the Synthesis of DGA Under Continuous Flow Conditions

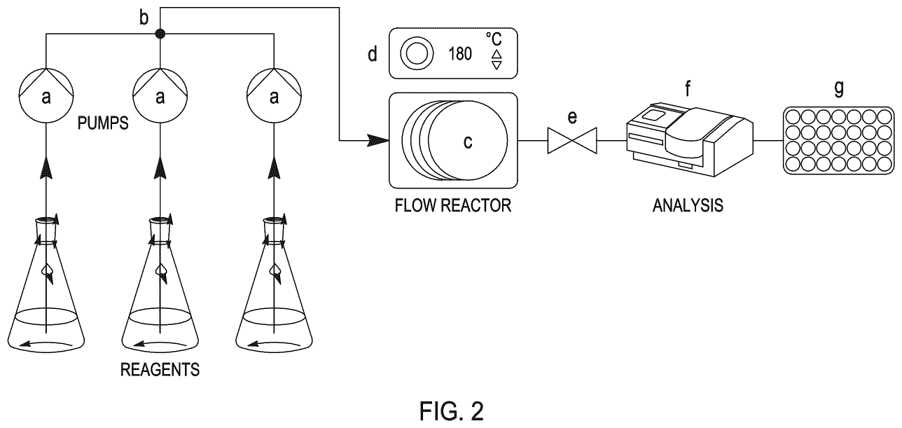

After the initial batch optimization of Example 1 was completed, continuous flow optimization was explored. A diagram of the continuous flow system used is shown in FIG. 2. The continuous flow system included a) three piston pumps for delivery of reactants and solvent, b) a quad mixer to combine and mix reactants and solvent, c) a tubular or coil reactor for providing residence time for the reaction, d) temperature controller and heating tape for controlling reaction temperature, e) back pressure valve for controlling pressure of the reactor, f) infrared (IR) spectrometer equipped with flow cell for analysis, and g) fraction collector for the final reaction solution collection. Although three pumps are shown in FIG. 2, in various aspects of the present disclosure the number of pumps used can vary based on the experimental study (e.g., four, three, two, or one pump).

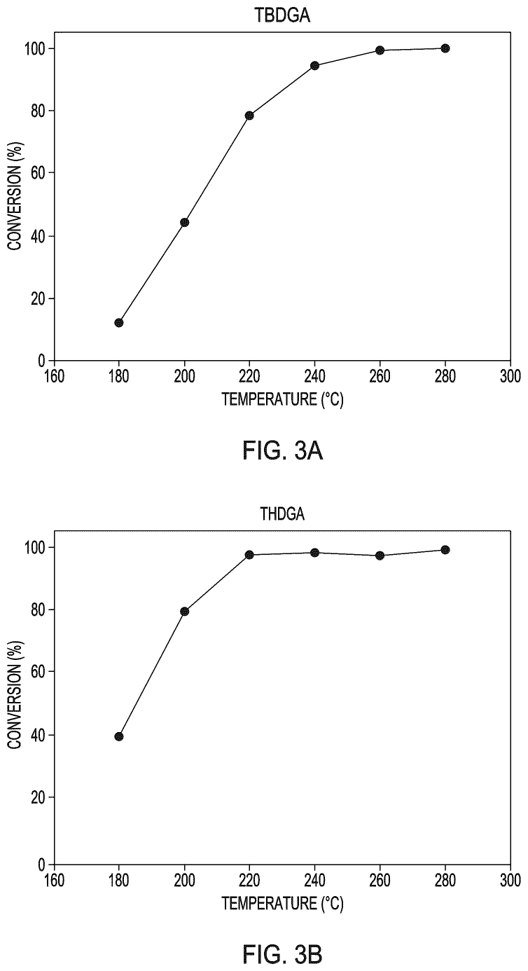

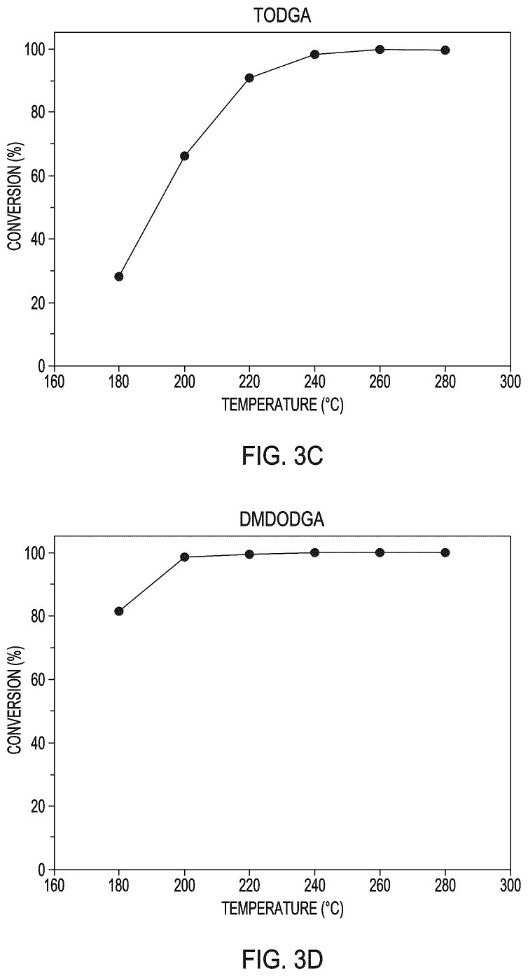

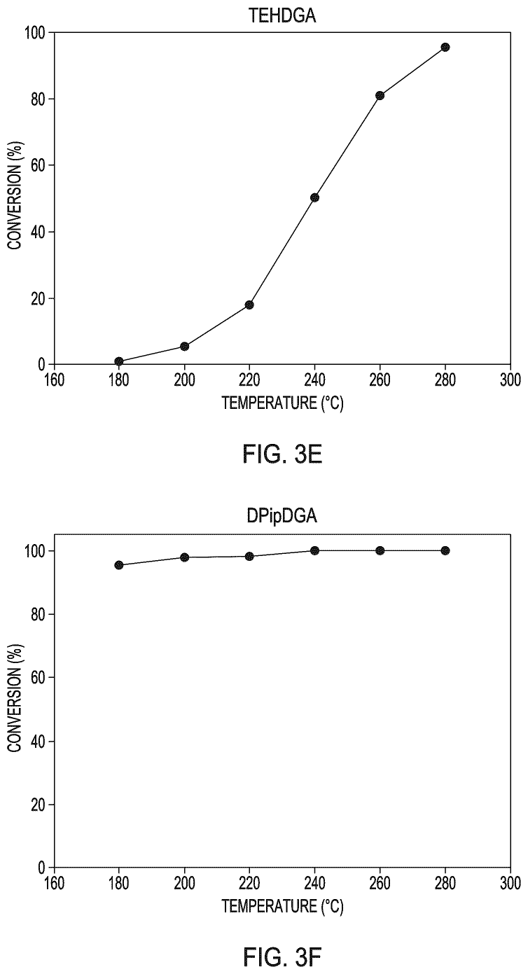

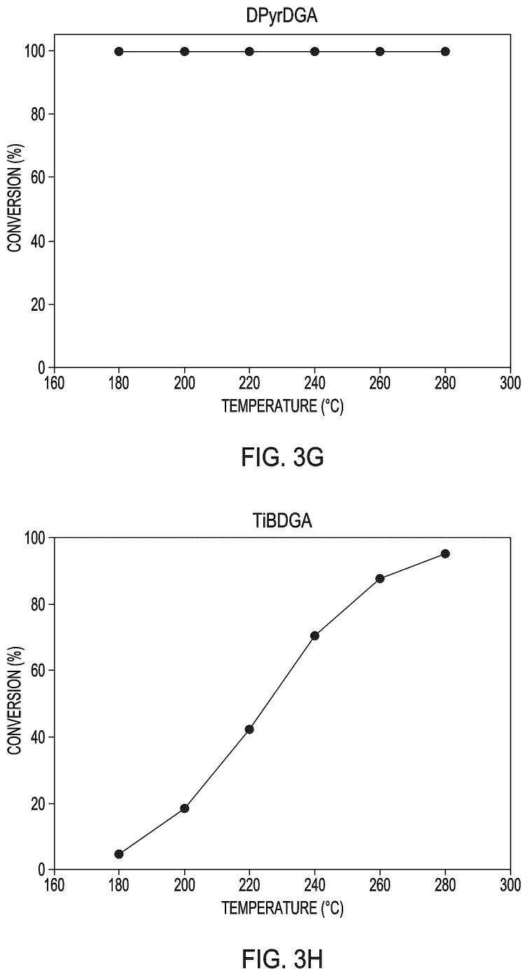

The three pumps were used to deliver dimethyl 2,2′-oxydiacetate (0.593 M) solution in xylene, neat dibutyl amine, and pure xylene at desired flow rates before being combined into one stream through a quad mixer. The mixed reaction solution was then injected into a 10.0 mL coil reactor (stainless steel, 1/16′ OD), which was wrapped with a heating tape controlled by a temperature regulator (OMEGA). The outlet of the coil reactor was connected to a 250 psi back pressure valve, which was further introduced into the flow cell of the IR spectrometer. Real-time reaction monitoring was achieved by accessing reactant conversion and production yield using online IR spectroscopy. The reaction solution was collected with an interval of 30 min into 7 mL test tubes using a programmed fraction collector. For reaction optimization, temperature (200, 220, and 240° C.) and the equivalents of dibutyl amine 2 (2, 4, 6, and 8 equivalents) were varied systematically. A total of 12 combinations of reaction parameters were run with a run time of 3 h each. The flow rate of 2,2′-oxydiacetate 1 solution was set constantly to 0.05 mL/min, and the flow rate of dibutyl amine changed from 0.01 to 0.04 mL/min, with the variation of xylene flow rate to complement the total flow rate to 1.0 mL/min. The overall reaction sequence ran smoothly with online IR analysis for 36 hours in total, giving the highest conversion over 99% over only 12 steps of optimization, as shown in FIGS. 3A-3H.

### Example 4. Continuous Synthesis of DGAs with Variation of Starting Materials

Scheme 2 illustrates the formation of diglycolamides 3 from diglycolic acid ester 1 and amine 2. The reactor setup was based on the reactor setup of Example 3. The continuous flow system for substrate scope studies included a) piston pump(s) for delivery of reactants and solvent, b) temperature controller and heating tape for controlling reaction temperatures, c) a coil reactor for providing residence time for the reaction, d) infrared (IR) spectrometer equipped with flow cell for analysis, e) back pressure valve for controlling pressure of the reactor, and f) fraction collector for the final reaction solution collection.

The pump was used to deliver a reaction mixture of dimethyl 2,2′-oxydiacetate (0.2 M) and dialkyl amine (1.6 M, 8 equiv.) in xylene at a flow rate of 0.2 mL/min. The reaction solution was then injected into a 12.0 mL coil reactor (stainless steel, ⅛′ OD), which was wrapped with a heating tape controlled by a temperature regulator (OMEGA). The outlet of the coil reactor was introduced into the flow cell of the IR spectrometer, which was further connected to a 450 psi back pressure regulator. Real-time reaction monitoring was achieved by accessing reactant conversion and production yield using online IR spectroscopy. The reaction solution was collected with an interval of 20 min into 7 mL test tubes using a programmed fraction collector. For reaction optimization, a total of 7 temperatures (180, 200, 220, 240, 260, and 280° C.) were investigated with a run time of 90 min each. The flow rate of the reaction solution was set constantly to 0.2 mL/min.

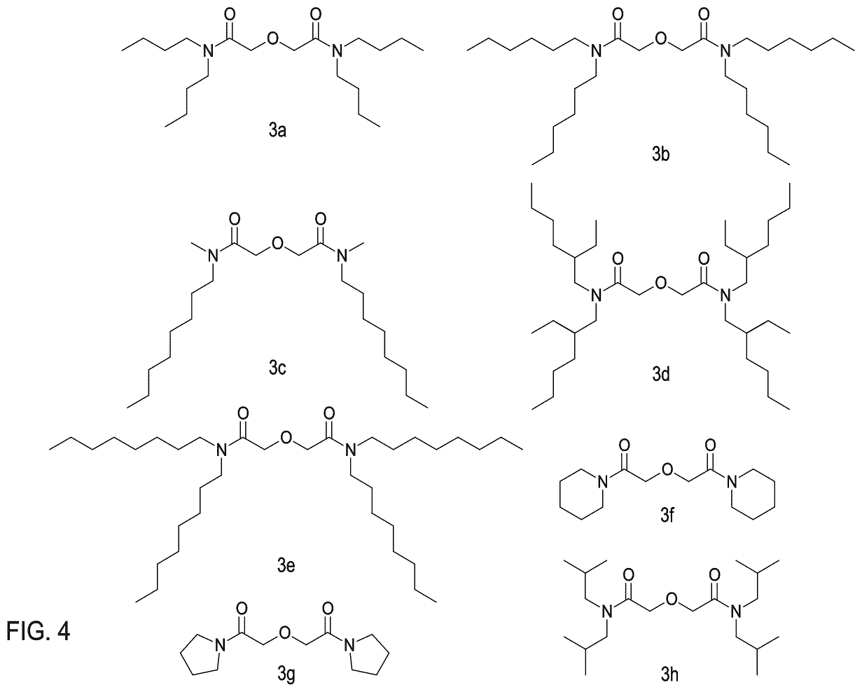

The same starting conditions were used as determined as optimal in Example 3. The reaction proceeded smoothly with various dialkyl amines. FIG. 4 illustrates structures of the diglycolamides formed via the continuous synthesis, which included TBDGA (3a), THDGA (3b), DMDODGA (3c), TEHDGA (3d), TODGA (3e), DPipDGA (3f), DPyrDGA (3g), TⁱBDGA (3h). The conversions are shown in Table 2. The production of the three most used DGAs, TEHDGA, TODGA, and DMDODGA, demonstrates the practical utility of the continuous flow synthesis.

TABLE 2
Conversion of diglycolamides formed via continuous synthesis.

| Diglycolamide | Conversion (%) |
|---|---|
| TBDGA (3a) | 99 |
| THDGA (3b) | 98 |
| DMDODGA (3c) | 100 |
| TEHDGA (3d) | 95 |
| TODGA (3e) | 98 |
| DPipDGA (3f) | 100 |
| DPyrDGA (3g) | 100 |
| TBDGA (3h) | 90 |

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects.

The following exemplary aspects are provided, the numbering of which is not to be construed as designating levels of importance:

Aspect 1 provides a method of forming a diglycolamide, the method comprising:
heating a reaction solution comprising
   a diglycolic acid ester having the structure R⁵O—C(O)—CH(R³)—O—CH(R⁴)—C(O)—OR⁶, and
   an amine having the structure R₁—NH—R²,
to form the diglycolamide having the structure (R¹)(R²)N—C(O)—CH(R³)—O—CH(R⁴)—C(O)—N(R¹)(R²);
wherein
   R⁵ and R⁶ are independently chosen from a substituted or unsubstituted (C₁-C₃₀)hydrocarbyl optionally interrupted by 0, 1, 2, or 3 groups independently chosen from —O— and —S—,
   R¹ and R² are independently chosen from a substituted or unsubstituted (C₁-C₃₀)hydrocarbyl optionally interrupted by 0, 1, 2, or 3 groups independently chosen from —O— and —S—, or wherein R¹ and R² together form a ring containing the nitrogen atom to which they are attached and R¹ and R² are together a (C₄-C₁₂)hydrocarbylene optionally interrupted by 0, 1, 2, or 3 groups independently chosen from —O— and —S—, and
   R³ and R⁴ are independently chosen from —H and a substituted or unsubstituted (C₁-C₃₀)hydrocarbyl optionally interrupted by 0, 1, 2, or 3 groups independently chosen from —O— and —S—.

Aspect 2 provides the method of Aspect 1, wherein R⁵ and R⁶ are independently chosen from (C₁-C₃₀)hydrocarbyl.

Aspect 3 provides the method of any one of Aspects 1-2, wherein R⁵ and R⁶ are independently chosen from (C₁-C₁₀)alkyl.

Aspect 4 provides the method of any one of Aspects 1-3, wherein R⁵ and R⁶ are independently chosen from methyl, ethyl, propyl, (C₄)alkyl, (C₅)alkyl, (C₆)alkyl, (C₇)alkyl, (C₈)alkyl, (C₉)alkyl, and (C₁₀)alkyl.

Aspect 5 provides the method of any one of Aspects 1-4, wherein R⁵ and R⁶ are —CH₃.

Aspect 6 provides the method of any one of Aspects 1-5, wherein R³ and R⁴ are independently chosen from —H and (C₁-C₃₀)hydrocarbyl.

Aspect 7 provides the method of any one of Aspects 1-6, wherein R³ and R⁴ are independently chosen from —H and (C₁-C₁₀)alkyl.

Aspect 8 provides the method of any one of Aspects 1-7, wherein R³ and R⁴ are —H.

Aspect 9 provides the method of any one of Aspects 1-8, wherein the diglycolic acid ester is dimethyl 2,2′-oxydiacetate.

Aspect 10 provides the method of any one of Aspects 1-9, wherein R¹ and R² are independently chosen from (C₁-C₃₀)hydrocarbyl.

Aspect 11 provides the method of any one of Aspects 1-10, wherein R¹ and R² are independently chosen from (C₁-C₁₀)alkyl.

Aspect 12 provides the method of any one of Aspects 1-11, wherein R¹ and R² are independently chosen from methyl, ethyl, propyl, (C₄)alkyl, (C₅)alkyl, (C₆)alkyl, (C₇)alkyl, (C₈)alkyl, (C₉)alkyl, and (C₁₀)alkyl.

Aspect 13 provides the method of any one of Aspects 1-12, wherein the amine is chosen from dibutylamine, dihexylamine, dioctylamine, methyloctylamine, di(2-ethylhexyl)amine, pyrrolidine, and piperidine.

Aspect 14 provides the method of any one of Aspects 1-13, wherein the diglycolamide is chosen from tetrabutyl diglycolamide, tetrahexyl diglycolamide, tetraoctyl diglycolamide, dimethyldioctyl diglycolamide, tetra(2-ethylhexyl) diglycolamide, di(piperidin-1-yl) diglycolamide, di(pyrrolidin-1-yl) diglycolamide, and tetra(iso-butyl) diglycolamide.

Aspect 15 provides the method of any one of Aspects 1-14, wherein the reaction solution has a molar ratio of the amine to the glycolic acid of at least 2:1.

Aspect 16 provides the method of any one of Aspects 1-15, wherein the reaction solution has a molar ratio of the amine to the glycolic acid of 2:1 to 10:1

Aspect 17 provides the method of any one of Aspects 1-16, wherein the reaction solution has a molar ratio of the amine to the glycolic acid of 3:1 to 5:1.

Aspect 18 provides the method of any one of Aspects 1-17, wherein the reaction solution further comprises an organic solvent.

Aspect 19 provides the method of Aspect 18, wherein the organic solvent has a boiling point in the range of 100° C. to 300° C.

Aspect 20 provides the method of any one of Aspects 18-19, wherein the organic solvent has a boiling point in the range of 130° C. to 240° C.

Aspect 21 provides the method of any one of Aspects 18-20, wherein the organic solvent is an aprotic solvent.

Aspect 22 provides the method of any one of Aspects 18-21, wherein the organic solvent is toluene, 1,4-dioxane, ethylene glycol, DMF (dimethylformamide), ACN (acetonitrile), xylene, anisole, trifluoromethyl anisole, kerosene, or a combination thereof.

Aspect 23 provides the method of any one of Aspects 18-22, wherein a concentration of the diglycolic acid ester in the reaction solution is 0.01 M to 10 M.

Aspect 24 provides the method of any one of Aspects 18-23, wherein a concentration of the diglycolic acid ester in the reaction solution is 0.5 M to 1.5 M.

Aspect 25 provides the method of any one of Aspects 1-24, wherein the reaction solution further comprises an additive.

Aspect 26 provides the method of Aspect 25, wherein the additive is insoluble in the reaction during the heating of the reaction solution and during the formation of the diglycolamide.

Aspect 27 provides the method of any one of Aspects 25-26, wherein the additive comprises SiO₂, mesoporous silica, Al₂O₃, a material including a Lewis acid site, a material including a Bronsted acid site, a homogenous acid material, a heterogenous acid material, or a combination thereof.

Aspect 28 provides the method of any one of Aspects 1-27, wherein the reaction solution is substantially free of materials other than the diglycolic acid ester, the amine, any of the diglycolamide formed from the diglycolic acid ester and the amine, and one or more optional solvents.

Aspect 29 provides the method of any one of Aspects 1-28, wherein the method has a yield of the diglycolamide from the diglycolic acid ester of 50% to 100%.

Aspect 30 provides the method of any one of Aspects 1-29, wherein the method has a yield of the diglycolamide from the diglycolic acid ester of 90% to 100%.

Aspect 31 provides the method of any one of Aspects 1-30, wherein the method has a conversion of the diglycolic acid ester of 50% to 100%.

Aspect 32 provides the method of any one of Aspects 1-31, wherein the method has a conversion of the diglycolic acid ester of 90% to 100%.

Aspect 33 provides the method of any one of Aspects 1-32, wherein the method is a batch method.

Aspect 34 provides the method of any one of Aspects 1-33, wherein the method is a continuous method.

Aspect 35 provides the method of Aspect 34, wherein the reaction solution is flowed continuously through a continuous flow reactor.

Aspect 36 provides the method of Aspect 35, wherein the reactor comprises a heater.

Aspect 37 provides the method of any one of Aspects 1-36, wherein the heating comprises heating to a treatment temperature for a treatment duration.

Aspect 38 provides the method of Aspect 37, wherein the treatment temperature comprises 100° C. to 300° C.

Aspect 39 provides the method of any one of Aspects 37-38, wherein the treatment temperature comprises 180° C. to 280° C.

Aspect 40 provides the method of any one of Aspects 37-39, wherein the treatment duration comprises 1 min to 24 h.

Aspect 41 provides the method of any one of Aspects 37-40, wherein the treatment duration comprises 1 h to 12 h.

Aspect 42 provides the method of any one of Aspects 1-41, wherein the reaction solution is substantially free of halides.

Aspect 43 provides the method of any one of Aspects 1-42, wherein the reaction solution is substantially free of an acid halide of the diglycolic acid ester.

Aspect 44 provides the method of any one of Aspects 1-43, wherein the reaction solution is substantially free of the acid form of the diglycolic acid ester.

Aspect 45 provides the method of any one of Aspects 1-44, wherein the reaction solution is substantially free of thionyl halides.

Aspect 46 provides the method of any one of Aspects 1-45, further comprising using the diglycolamide as a ligand to perform an extraction and/or separation of rare earth metals, nuclear waste, toxic or hazardous materials, or a combination thereof.

Aspect 47 provides a method of forming a diglycolamide, the method comprising:
heating a reaction solution in a continuous flow reactor at a temperature of 180° C. to 280° C., the reaction solution comprising
   a diglycolic acid ester having the structure R⁵O—C(O)—CH₂—O—CH₂—C(O)—OR⁶, and
   an amine having the structure R¹—NH—R²,
to form the diglycolamide having the structure (R¹)(R²)N—C(O)—CH₂—O—CH₂—C(O)—N(R¹)(R²);
wherein
   R⁵ and R⁶ are independently chosen from (C₁-C₁₀)alkyl, and
   R¹ and R² are independently chosen from (C₁-C₁₀)alkyl, or wherein R¹ and R² together form a ring containing the nitrogen atom to which they are attached and R¹ and R² are together a (C₄-C₁₂)hydrocarbylene.

Aspect 48 provides a system for performing the method of any one of Aspects 1-47, the system comprising:
one or more pumps;
a mixer to combine materials flowed from the one or more pumps and to form the reaction solution;
a continuous flow reactor that receives the reaction solution from the mixer; and
an analyzer that receives the reaction solution from the continuous flow reactor.

Aspect 49 provides the system of Aspect 48, further comprising a processor that receives data from the analyzer and that controls the pumps and the continuous flow reactor.

Aspect 50 provides the system of Aspect 49, wherein the processor optimizes flow rate and reaction conditions with data from the analyzer for maximum conversion of starting materials and maximum yield of product.

Aspect 51 provides the method or system of any one or any combination of Aspects 1-50 optionally configured such that all elements or options recited are available to use or select from.

What is claimed is:

1. A method of forming a diglycolamide, the method comprising:
heating a reaction solution comprising
a diglycolic acid ester having the structure R⁵O—C(O)—CH(R³)—O—CH(R⁴)—C(O)—OR⁶, and
an amine having the structure R¹—NH—R²,
to form the diglycolamide having the structure (R¹)(R²)N—C(O)—CH(R³)—O—CH(R⁴)—C(O)—N(R¹)(R²);
wherein
R⁵ and R⁶ are independently chosen from a substituted or unsubstituted (C₁-C₃₀) hydrocarbyl optionally interrupted by 0, 1, 2, or 3 groups independently chosen from —O— and —S—,
R¹ and R² are independently chosen from a substituted or unsubstituted (C₁-C₃₀) hydrocarbyl optionally interrupted by 0, 1, 2, or 3 groups independently chosen from —O— and —S—, or wherein R¹ and R² together form a ring containing the nitrogen atom to which they are attached and R¹ and R² are together a (C₄-C₁₂) hydrocarbylene optionally interrupted by 0, 1, 2, or 3 groups independently chosen from —O— and —S—, and
R³ and R⁴ are independently chosen from —H and a substituted or unsubstituted (C₁-C₃₀) hydrocarbyl optionally interrupted by 0, 1, 2, or 3 groups independently chosen from —O— and —S—.

2. The method of claim 1, wherein R¹, R², R⁵ and R⁶ are independently chosen from methyl, ethyl, propyl, (C₄)alkyl, (C₅)alkyl, (C₆)alkyl, (C₇)alkyl, (C₈)alkyl, (C₉)alkyl, and (C₁₀)alkyl.

3. The method of claim 1, wherein R³ and R⁴ are —H.

4. The method of claim 1, wherein the diglycolic acid ester is dimethyl 2,2′-oxydiacetate.

5. The method of claim 1, wherein the amine is chosen from dibutylamine, dihexylamine, dioctylamine, methyloctylamine, di(2-ethylhexyl)amine, pyrrolidine, and piperidine.

6. The method of claim 1, wherein the diglycolamide is chosen from N,N,N′,N′-tetrabutyl diglycolamide (TBDGA), tetra-n-hexyl diglycolamide (THDGA), tetra-n-octyl diglycolamide (TODGA), N,N′-dimethyl-N,N′-dioctyl diglycolamide (DMDODGA), tetra(2-ethylhexyl) diglycolamide (TEHDGA), di(piperidin-1-yl) diglycolamide (DPipDGA), di(pyrrolidin-1-yl) diglycolamide (DPyrDGA), and tetra(iso-butyl) diglycolamide (TiBDGA).

7. The method of claim 1, wherein the reaction solution has a ratio of the amine to the glycolic acid of 3:1 to 5:1.

8. The method of claim 1, wherein the reaction solution further comprises an organic solvent.

9. The method of claim 8, wherein the organic solvent has a boiling point in the range of 100° C. to 300° C., and/or wherein the organic solvent is toluene, 1,4-dioxane, ethylene glycol, DMF, ACN, xylene, anisole, trifluoromethyl anisole, kerosene, or a combination thereof.

10. The method of claim 7, wherein a concentration of the diglycolic acid ester in the reaction solution is 0.5 M to 1.5 M.

11. The method of claim 1, wherein the reaction solution further comprises an additive, wherein the additive is insoluble in the reaction solution during the heating of the reaction solution and during the formation of the diglycolamide.

12. The method of claim 11, wherein the additive comprises SiO₂, mesoporous silica, Al₂O₃, a material including a Lewis acid site, a material including a Bronsted acid site, a homogeneous acid material, a heterogeneous acid material, or a combination thereof.

13. The method of claim 1, wherein the method has a yield of the diglycolamide from the diglycolic acid ester of 70% to 100% and a conversion of the diglycolic acid ester of 90% to 100%.

14. The method of claim 1, wherein the method is a batch method.

15. The method of claim 1, wherein the method is a continuous method, wherein the reaction solution is flowed continuously through a continuous flow reactor.

16. The method of claim 1, wherein the heating comprises heating to a treatment temperature for a treatment duration, wherein the treatment temperature comprises 100° C. to 300° C., and wherein the treatment duration comprises 1 min to 24 h.

17. The method of claim 1, further comprising using the diglycolamide as a ligand to perform an extraction and/or separation of rare earth metals, nuclear waste, toxic or hazardous materials, or a combination thereof.

18. A method of forming a diglycolamide, the method comprising:
heating a reaction solution in a continuous flow reactor at a temperature of 180° C. to 280° C., the reaction solution comprising
a diglycolic acid ester having the structure R⁵O—C(O)—CH₂—O—CH₂—C(O)—OR⁶,
an amine having the structure R¹—NH—R², and
an organic solvent,
to form the diglycolamide having the structure (R¹)(R²)N—C(O)—CH₂—O—CH₂—C(O)—N(R¹)(R²);
wherein
R⁵ and R⁶ are independently chosen from (C₁-C₁₀)alkyl, and
R¹ and R² are independently chosen from (C₁-C₁₀)alkyl, or wherein R¹ and R² together form a ring containing the nitrogen atom to which they are attached and R¹ and R² are together a (C₄-C₁₂) hydrocarbylene.

19. A system for performing the method of claim 1, the system comprising:
one or more pumps;
a mixer to combine materials flowed from the one or more pumps and to form the reaction solution;
a continuous flow reactor that receives the reaction solution from the mixer; and
an analyzer that receives the reaction solution from the continuous flow reactor.

20. The system of claim 19, further comprising a processor that receives data from the analyzer and that controls the pumps and the continuous flow reactor, wherein the processor optimizes flow rate and reaction conditions with data from the analyzer for maximum conversion of starting materials and maximum yield of product.
